(19) United States
(12) Patent Application Publication
(10) Pub. No.: US 2020/0166526 A1
(43) Pub. Date: May 28, 2020

(54) METHODS FOR DIAGNOSING AND TREATING ALZHEIMER'S DISEASE

(71) Applicant: William Beaumont Hospital, Royal Oak, MI (US)

(72) Inventors: Stewart Graham, Berkley, MI (US); Ray Bahado-Singh, Grosse Pointe Shores, MI (US); Michael Maddens, Sterling Heights, MI (US); Ali Yilmaz, Royal Oak, MI (US)

(21) Appl. No.: 16/633,929

(22) PCT Filed: Jul. 24, 2018
(86) PCT No.: PCT/US18/43499

§ 371 Date: Jan. 24, 2020

Related U.S. Application Data
(60) Provisional application No. 62/536,728, filed on Jul. 25, 2017.

(51) Int. Cl. G01N 33/68; A61P 25/28; A61K 31/13; A61K 31/27; A61K 31/445; A61K 31/55; G01N 33/70; G01N 33/66

(52) U.S. Cl. CPC G01N 33/6896; A61P 25/28; A61K 31/13; A61K 31/27; G01N 2800/52; A61K 31/55; G01N 33/70; G01N 33/66; G01N 2800/2821; A61K 31/445

(57) ABSTRACT

The present invention relates to methods for diagnosing and/or treating Alzheimer's Disease (AD) by detecting in a human sample the levels of one or more of the metabolites galactose, imidazole acetone, creatinine, and 5-aminopentanoate; diagnosing the patient with AD when one or more of the metabolites galactose, imidazole acetone, creatinine, and 5-aminopentanoate in the sample is at a different level than a statistically validated threshold for the respective metabolite. In some embodiments, the AD is diagnosed and the patient is treated for AD.

### CROSS-REFERENCE TO RELATED APPLICATIONS

This application claims priority to U.S. Provisional Application 62/536,728 filed on Jul. 25, 2017, the disclosure of which is considered part of the disclosure of this application and is hereby incorporated by reference in their entireties.

### FIELD OF THE INVENTION

The present invention is in the field of biochemistry and medicine and relates to methods for diagnosing and treating Alzheimer's Disease.

### BACKGROUND OF THE INVENTION

Alzheimer's disease (AD) is a neurodegenerative disorder characterized by the accumulation of amyloid-β plaques and tau tangles. “Mild cognitive impairment” (MCI) is a progressive disease that is diagnosed when loss of memory and critical thinking skills are beyond what would be expected for an individual given their age and educational background; but the loss is not so severe as to merit a diagnosis of dementia. MCI is thought to be a transitional state between normal aging and AD. The conversion rate from MCI to AD is estimated to be approximately 10% per year and that number increases every year.

Currently, one of the problems with treatments for Alzheimer's disease (AD) is that irreversible brain damage already may have occurred before a clinical diagnosis is provided. Current therapies for AD are initiated after diagnosis. Early identification of the disease is of paramount importance as the majority of clinical trials fail due to mis-stratification of patients.

There is a need for biomarkers to facilitate more efficacious drug development and clinical trials, and for treatment for AD and MCI. Valid and reliable biomarkers for AD and MCI not only aid clinicians in recognizing the disease in its earliest symptomatic stages, but are important for disease prevention and AD (and MCI) therapies. Sensitive biomarkers can permit intervention before substantial neuropathological damage has occurred and before the manifestation of dementia.

### SUMMARY OF THE INVENTION

Using ¹H NMR metabolomics, the inventors profiled saliva samples collected from healthy-controls (n=12), mild cognitive impairment sufferers (n=8) and Alzheimer's Disease patients (n=9); and identified significant concentration changes in metabolites in the saliva of MCI and AD patients compared to controls. This application describes using these metabolites in for the diagnosis of AD and MCI, including early diagnosis, and in combination with treatments for AD or MCI (to retard, inhibit, or reverse the progression of MCI or AD). Given the ease and convenience of collecting saliva, the inventive biomarkers detected in saliva are ideal for screening those at risk of developing AD. Use of these biomarkers allows for identification early-on of those individuals: at risk of developing MCI or AD; having MCI or AD; having MCI which may covert to AD; and in need of treatment for AD or MCI.

One embodiment of the invention is a method of treating Alzheimer's Disease (AD) in a human patient, the method including obtaining a sample from the human patient, wherein the sample consists of one or more of the metabolites galactose, imidazole, acetone, creatinine, and 5-aminopentanoate; detecting a level of one or more of the metabolites acetone, creatinine, and 5-aminopentanoate in the sample; and administering a therapeutically effective amount of a treatment for AD to the patient when the level of one or more of the metabolites galactose, imidazole, acetone, creatinine, and 5-aminopentanoate in the sample is at a different level than a statistically validated threshold for the respective metabolite.

In one example, the treatment may be an acetylcholinesterase inhibitor or an N-methyl-D-aspartate receptor (NMDAR) antagonist, or a combination thereof. Further, the acetylcholinesterase inhibitor may be rivastigmine, donepezil, and/or galantamine; and the NMDAR antagonist may be memantine. In a further aspect, the sample may be saliva. Additionally, the one or more of the metabolites may be acetone, creatinine, and/or 5-aminopentanoate (or creatinine and/or 5-aminopentanoate), and the different level may be an elevated level of the one or more metabolites in the sample as compared to the statistically validated threshold for the respective metabolite.

In a further aspect, the method also may include performing one or more cognitive tests on the patient; and treating the patient for AD when (a) the level of one or more of the metabolites galactose, imidazole, acetone, creatinine, and 5-aminopentanoate in the sample is at a different level than a statistically validated threshold for the respective metabolite; and (b) the one or more cognitive tests indicates AD. The one of more cognitive tests may be a memory test, a problem solving test, an attention test, a counting test, and/or a language abilities test.

Another embodiment of the invention is a method of treating a human patient at risk for developing Alzheimer's Disease (AD), the method including: obtaining a sample from the human patient, wherein the sample includes one or more of the metabolites selected from galactose, imidazole, acetone, creatinine, and 5-aminopentanoate; detecting a level of the one or more metabolites galactose, imidazole, acetone, creatinine, and 5-aminopentanoate in the sample; and administering a therapeutically effective amount of a treatment for AD to the patient to slow or prevent the onset of AD symptoms when the level of one or more of the metabolites galactose, imidazole, acetone, creatinine, and 5-aminopentanoate in the sample is at a different level than a statistically validated threshold for the respective metabolite. The treatment may be an acetylcholinesterase inhibitor or an N-methyl-D-aspartate receptor (NMDAR) antagonist, or a combination thereof. And if the treatment is an acetylcholinesterase inhibitor, it may be rivastigmine, donepezil, or galantamine. If the treatment is an NMDAR antagonist it may be memantine. In a further aspect, the sample may be saliva.

In one example, the one or more of the metabolites may be acetone, creatinine, or 5-aminopentanoate, and the different level may be an elevated level of the one or more metabolites in the sample than the statistically validated threshold for the respective metabolite. Further, the one or more of the metabolites may be creatinine and/or 5-aminopentanoate.

A further embodiment of the invention is a method of treating Alzheimer's Disease (AD) in a human patient, the method including: obtaining a sample from the human patient, wherein the sample includes one or more of the metabolites galactose, imidazole acetone, creatinine, and 5-aminopentanoate; requesting testing for a level of one or more of the metabolites galactose, imidazole acetone, creatinine, and 5-aminopentanoate in the sample; and administering a therapeutically effective amount of a treatment for AD to the patient when the level of one or more of the metabolites galactose, imidazole acetone, creatinine, and 5-aminopentanoate in the sample is at a different level than a statistically validated threshold for the respective metabolite. In one example, the treatment may be an acetylcholinesterase inhibitor (which may be rivastigmine, donepezil, and galantamine) and/or an N-methyl-D-aspartate receptor (NMDAR) antagonist (e.g., memantine), or a combination thereof. And the sample may be saliva.

In one example, the one or more of the metabolites are acetone, creatinine, and/or 5-aminopentanoate (e.g., creatinine and/or 5-aminopentanoate), and the different level may be an elevated level of the one or more metabolites in the sample than the statistically validated threshold for the respective metabolite.

The inventive method also may include: requesting one or more cognitive tests of the patient; and treating the patient for AD when (a) the level of one or more of the metabolites galactose, imidazole, acetone, creatinine, and 5-aminopentanoate in the sample is at a different level than a statistically validated threshold for the respective metabolite; and (b) the one or more cognitive tests indicates AD. The one of more cognitive tests may include a memory test, a problem solving test, an attention test, a counting test, and/or a language abilities test.

In one aspect, the invention includes a method of treating Alzheimer's Disease (AD) in a human patient, the method including: obtaining a sample from the human patient, wherein the sample includes two or more of galactose, imidazole, acetone, creatinine, and 5-aminopentanoate, and proprionate; detecting a level of the two or more of galactose, imidazole, acetone, creatinine, and 5-aminopentanoate, and proprionate in the sample; and administering a therapeutically effective amount of a treatment for AD to the patient when the level of two or more of galactose, imidazole, acetone, creatinine, and 5-aminopentanoate, and proprionate in the sample are at a different level than a statistically validated threshold for the respective metabolites. Here, the treatment may be an acetylcholinesterase inhibitor or an N-methyl-D-aspartate receptor (NMDAR) antagonist, or a combination thereof; and the sample may be saliva.

In another aspect, the invention is a method of treating a mild cognitive impairment (MCI) in a human patient, the method including: obtaining a sample from the human patient, wherein the sample includes one or more of the metabolites selected from galactose, imidazole, and acetone; detecting a level of one or more of the metabolites galactose, imidazole, and acetone in the sample; and administering a therapeutically effective amount of a treatment for MCI to the patient when the level of one or more of the metabolites galactose, imidazole, and acetone in the sample is at a different level than a statistically validated threshold for the respective metabolite.

In a further aspect, the invention is a method for diagnosing Alzheimer's Disease in a human patient, the method including: obtaining a sample from the human patient, wherein the sample consists of one or more of the metabolites galactose, imidazole, acetone, creatinine, and 5-aminopentanoate; detecting a level of one or more of the metabolites galactose, imidazole, acetone, creatinine, and 5-aminopentanoate in the sample; and diagnosing Alzheimer's Disease in the patient when the level of one or more of the metabolites galactose, imidazole, acetone, creatinine, and 5-aminopentanoate in the sample is at a different level than a statistically validated threshold for the respective metabolite. The sample may be saliva. Further, the one or more of the metabolites may be acetone, creatinine, and 5-aminopentanoate, and the different level may be an elevated level of the one or more metabolites in the sample than the statistically validated threshold for the respective metabolite.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings, certain embodiment(s) which are presently preferred. It should be understood, however, that the invention is not limited to the precise arrangements and instrumentalities shown.

FIGS. 1A-1C: Receiver operating characteristics (ROC) curve analysis of metabolite data. All models were validated following a 10-fold cross validation.

FIG. 1A shows the logistic regression ROC analysis for controls vs MCI sufferers (AUC: 0.826 (0.634-1.000), Sensitivity: 0.909 (0.909-1.000), Specificity: 0.897 (0.707-1.000).

FIG. 1B shows the logistic regression ROC analysis of MCI sufferers vs. AD (AUC: 0.871 (0.689-1.000), Sensitivity: 0.909 (0.909-1.000), Specificity: 0.842 (0.678-1.000.

FIG. 1C shows the logistic regression ROC analysis for controls vs. AD (AUC: 0.897 (0.707-1.000), Sensitivity: 0.900 (0.900-1.000), Specificity: 0.944 (0.839-1.000)).

### DETAILED DESCRIPTION OF THE INVENTION

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

All references, patents, patent publications, articles, and databases, referred to in this application are incorporated herein by reference in their entirety, as if each were specifically and individually incorporated herein by reference. Such patents, patent publications, articles, and databases are incorporated for the purpose of describing and disclosing the subject components of the invention that are described in those patents, patent publications, articles, and databases, which components might be used in connection with the presently described invention. The information provided below is not admitted to be prior art to the present invention, but is provided solely to assist the understanding of the reader.

The details of one or more embodiments of the invention are set forth in the accompanying drawings and the description below. Other features, embodiments, and advantages of the invention will be apparent from the description and drawings, and from the claims. The preferred embodiments of the present invention may be understood more readily by reference to the following detailed description of the specific embodiments and the Examples included hereafter.

For clarity of disclosure, and not by way of limitation, the detailed description of the invention is divided into the subsections that follow.

Unless defined otherwise, all technical and scientific terms used herein have the meaning commonly understood by one of ordinary skill in the art to which this invention belongs. Generally, the nomenclature used herein and the laboratory procedures in cell culture, molecular genetics, organic chemistry and protein chemistry described below are those well-known and commonly employed in the art. Although any methods, devices and materials similar or equivalent to those described herein can be used in the practice or testing of the invention, the inventive methods, devices and materials are now described.

### Definitions

In this specification and the appended claims, the singular forms “a,” “an” and “the” include plural reference unless the context clearly dictates otherwise.

As used in the application, “administering”, when used in conjunction with a treatment means providing or performing medical services with respect to a subject in need of a treatment. For example, when used when used in conjunction with a therapeutic, administering means to deliver a therapeutic directly into or onto a subject's tissue or to administer a therapeutic to a subject whereby the therapeutic positively impacts the tissue or subject. “Administering” a composition may be accomplished by oral administration, injection, infusion, absorption (e.g., transdermal patch) or by any method in combination with other known techniques. “Administering” may include the act of self-administration or administration by another person such as, for example, a healthcare provider or other individual.

The terms “Alzheimer's Disease” or “AD” refer to a form of dementia, which includes loss of critical thinking, cognitive functioning (e.g., memory loss), and/or behavioral abilities.

The term “data set” refers to metabolite level data collected from a plurality of patient samples and control samples. The term “training data set” refers to a data set used to train a machine-learning algorithm. The term “subset” refers to a portion of a data set. A subset may be metabolite level data for a single patient sample or control sample or a subset may be metabolite level data for a small number of samples, whether control, patient or both. A subset may be a random grouping of one or more control and patient samples.

The terms “diagnosis” or “diagnosing” when used in connection with AD mean a determination (by one or more individuals) that the cause or nature of a problem, situation, or condition in a subject is AD, or a confirmation of the diagnosis of the disease that includes alternative or companion AD diagnostics, other signs and/or symptoms (e.g., diagnosing based in whole or in part on the level(s) of the AD-indicating metabolites described herein). The terms “diagnosis” or “diagnosing” when used in connection with MCI is used to mean a determination (by one or more individuals) that the cause or nature of a problem, situation, or condition in a subject is MCI, or a confirmation of the diagnosis of the disease that includes alternative or companion MCI diagnostics, other signs and/or symptoms (e.g., diagnosing based in whole or in part on the level(s) of the MCI-indicating metabolites described herein). A “diagnosis” of AD or MCI may include a test or an assessment of the degree of disease severity (e.g., “mild,” “moderate,” or “severe”), current state of disease progression (e.g., “early”, “middle,” or “late” stages of AD or MCI), or include a comparative assessment to an earlier diagnosis (e.g., the AD's or MCI's symptoms are advancing, stable, or in remission). A diagnosis may include a “prognosis,” that is, a future prediction of the progression of AD or MCI, based on the observed disease state (e.g., based in whole or in part on the different level(s) of the one or more AD-indicating or MCI-indicating metabolites described herein). A diagnosis or prognosis may be based on one or more samples obtained from a subject, and may involve a prediction of disease response to a particular treatment or combination of treatments for AD or MCI.

The terms “level” or “metabolite level” refer to a quantifiable amount of a metabolite in a sample. For example, the level may be a concentration level from an assay for a metabolite such as galactose, imidazole, acetone, creatinine, 5-aminopentanoate or proprionate. The level of the metabolite in a sample may be expressed in wt %, vol %, mol % or the like, which may be calculated from data from an assay and may be based on calibration data. A “different level” or “elevated level” of a metabolite refers to the amount or concentration of a metabolite in a sample from a patient compared to statistically validated thresholds, e.g., the amount of the metabolite in a sample(s) from individual(s) that do not have AD, have AD (or a particular severity or stage of AD), have no symptoms of AD, or have other reference diseases. A “different level” or “elevated level” of a metabolite also may refer to the amount or concentration of a metabolite in a sample from a patient compared to statistically validated thresholds, e.g., the amount of the metabolite in a sample(s) from individual(s) that do not have MCI, have MCI (or a particular severity or stage of MCI), have no symptoms of MCI, or have other reference diseases. For example, a metabolite has an “elevated level” in the saliva from a subject when the metabolite is present at a higher concentration in the subject's saliva sample than in saliva from a subject who does not have AD. As a further example, for the metabolites acetone, creatinine, and 5-aminopentanoate, and proprionate, elevated levels in a saliva sample indicate the presence of or a risk for AD. Also, for the metabolites acetone and imidazole, elevated levels in a saliva sample indicate the presence of or a risk for MCI.

The term “sample” refers to a biological sample from a subject with a diagnosis of AD, or a biological sample from a subject at risk for AD or in need of diagnosis for AD where it is unknown whether the subject has AD, or a sample from a subject suspected of having AD. The term “sample” also refers to a biological sample from a subject with a diagnosis of MCI, or a biological sample from a subject at risk for MCI or in need of diagnosis for MCI where it is unknown whether the subject has MCI, or a sample from a subject suspected of having MCI. In one example, a patient sample may be a tissue, a solid, a gas or a fluid sample. Examples of a fluid sample include saliva, blood, serum, cerebrospinal fluid, synovial fluid, lymph, urine or plasma obtained from a patient. The term “control sample” refers to a sample from a subject either known not to have AD or not known to have AD because no symptoms of AD have presented or been observed. The term “control sample” also refers to a sample from a subject either known not to have MCI or not known to have MCI because no symptoms of MCI have presented or been observed.

The term “subject” or “patient” as used herein generally refer to any living organism and may include, but are not limited to, any human, primate, or non-human mammal in need of diagnosis and/or treatment for a condition, disorder or disease (e.g., Alzheimer's Disease). A “subject” may or may not be exhibiting the signs, symptoms, or pathology of AD or MCI at any stage of any embodiment.

The term “therapeutically effective amount” refers to the amount of treatment (e.g., of an active agent or pharmaceutical compound or composition) that elicits a biological and/or medicinal response in a patient, subject, tissue, or system that is being sought by a researcher, medical doctor or other clinician, or any combination thereof. A biological or medicinal response may include, for example, one or more of the following: (1) preventing a disorder, disease, or condition in an individual that may be predisposed to the disorder, disease, or condition but does not yet experience or display pathology or symptoms of the disorder, disease, or condition, (2) inhibiting a disorder, disease, or condition in an individual that is experiencing or displaying the pathology or symptoms of the disorder, disease, or condition or arresting further development of the pathology and/or symptoms of the disorder, disease, or condition, and/or (3) ameliorating a disorder, disease, or condition in an individual that is experiencing or exhibiting the pathology or symptoms of the disorder, disease, or condition or reversing the pathology and/or symptoms disorder, disease, or condition experienced or exhibited by the individual.

The term “treatment” or “treating” as used herein refers to administrating a medicine or the performance of medical procedures with respect to a subject, for either prophylaxis (prevention) or to cure or reduce the extent of or likelihood of occurrence or recurrence of an infirmity, malady, condition, symptom, or event in the instance where the subject is afflicted or is pre-disposed to becoming afflicted. As related to the present invention, the term may mean administrating medicine or the performance of medical procedures as therapy, prevention or prophylaxis of Alzheimer's Disease or the symptoms of Alzheimer's Disease. As related to the present invention, the term also may also mean administrating medicine or the performance of medical procedures as therapy, prevention or prophylaxis of MCI or the symptoms of MCI.

The inventors here present the results of an ¹H NMR-based metabolomics study discriminating MCI sufferers, AD patients, and healthy controls from each other. They analyzed specimens collected from AD patients, MCI sufferers, and corresponding age- and gender-matched controls. As described in the Examples below, the results demonstrate that there are significant differences in the concentrations of salivary metabolites in AD and MCI versus unaffected controls. Similarly, differences were found when the AD and the MCI groups were compared.

Based on the metabolite concentration data, the inventors were able to generate regression models that significantly differentiated both MCI and AD cases from controls. The regression model with the greatest predictive ability was created when separating controls from MCI using the concentrations of galactose, imidazole and acetone with an AUC=0.826 (95% CI: 0.634-1.00) and with a sensitivity and specificity of 0.909 and 0.897, respectively. When the concentration values of creatinine and 5-aminopentanoate from MCI sufferers versus AD patients were analyzed using logistic regression, an AUC value of 0.871 (0.689˜1.000) with a sensitivity and specificity of 0.909 and 0.842 was achieved. Further, the logistic regression model based on the concentration values of propionate and acetone for separating controls from AD patients produced an AUC=0.897 0.707-1.000) along with 0.900 sensitivity and 0.944 specificity. See, Table 1.

TABLE 1
The performance values for the ROC and the logistic regression
analysis.

| | | Best (Sensitivity + Specificity) | | |
|---|---|---|---|---|
| Models | AUC (95% CI) | Sensitivity | Specificity | Threshold |
| Control vs MCI | 0.826 (0.634-1.000) | 0.909 | 0.897 | 0.399 |
| MCI vs AD | 0.871 (0.689-1.000) | 0.909 | 0.842 | 0.404 |
| Control vs AD | 0.897 (0.707-1.000) | 0.900 | 0.944 | 0.223 |

Galactose is a monosaccharide sugar having the molecular formula C₆H₁₂O₆. Galactose is a simple carbohydrate that exists in both open-chain and cyclic form and is composed of the same elements as glucose (but has a different arrangement of atoms).

Imidazole is a heterocyclic aromatic organic compound having the molecular formula C₃H₄N₂. Imidazole is a planar 5-membered ring and it exists in two equivalent tautomeric forms.

Acetone (systematically named propanone) is an organic compound having the formula (CH₃)₂CO. Acetone is the simplest representative of the ketones.

Creatinine (2-amino-1-methyl-1H-imidazol-4-ol) is a compound having the molecular formula C₄H₇N₃O. Creatinine is produced by the metabolism of creatine.

5-aminopentanoate is an amino fatty acid anion that is the conjugate base of 5-aminopentanoic acid. 5-aminopentanoate has the molecular formula C₅H₁₀NO₂.

Propionate is the conjugate base of propionic acid and has the molecular formula C₃H₅O₂_{^{−}}.

The inventors have surprisingly found that by detecting one or more of the metabolites galactose, imidazole, acetone, propionate, creatinine or 5-aminopentanoate, it is possible to diagnose and/or treat MCI or AD; by detecting one or more of acetone, propionate, creatinine or 5-aminopentanoate, it is possible to diagnose and/or treat AD; by detecting one or more of the metabolites, creatinine and/or 5-aminopentanoate, it is possible to use these metabolites as predictive biomarkers to differentiate between AD and MCI and/or thereby diagnose and/or treat AD; and by detecting one or more of the metabolites galactose, imidazole, and acetone, it is possible to diagnose and/or treat MCI. The inventors also have found that by detecting two or more of the metabolites proprionate, galactose, imidazole, acetone, propionate, creatinine or 5-aminopentanoate, it is possible to diagnose and/or treat MCI or AD. The detection and analysis of combinations of these metabolite levels may result in a synergistic effect, increasing the specificity and accuracy of diagnosis of AD. In some embodiments, the detection levels of the metabolites can be used to create a risk score based on algorithms generated using a training data set with levels of the metabolites from patient and control samples.

Patient Sample

The inventors have also found that the levels of the metabolites galactose, imidazole, acetone, propionate, creatinine and 5-aminopentanoate can be detected and measured in a patient sample. Given the convenience and the frequency with which it can be obtained, saliva may be used as the sample for the present inventive method.

Saliva is a clear, watery biofluid produced by the salivary glands to protect and lubricate the oral cavity. The chemical composition of saliva changes quite dramatically in response to a variety of different physiological states, stimuli, insults and stressors making it a good candidate for monitoring biological responses of the body to any directed case. This is because, many of the components in serum and cerebrospinal fluid (CSF) pass into the saliva via the blood by transcellular, intracellular, paracellular or extracellular routes involving passive diffusion or active transport within the salivary glands and the gingival sulcus. Unlike blood and cerebrospinal fluid, saliva is easily available through non-invasive means and represents a constant source of material for diagnostics.

Methods of detection, diagnosis, and/or treatment of the present invention include obtaining a sample. The sample may be a fluid from the patient, such as a fresh saliva sample that is totally unprocessed. In some embodiments, the sample is a preserved sample. The preserved sample comprises a preservative combined with the sample from the patient. The preservative may be any preservative known to preserve metabolites in the sample. That is, the preservative will inhibit significant degradation of the metabolites in the sample. More specifically, in one embodiment, the preservative will inhibit between from about 20% to about 100% (e.g., from about 40% to about 80% or from about 60% to about 100%) of the metabolite degradation that would occur if no preservative were combined with the sample. The preservative may be a commercially available saliva preservative. The sample may also be preserved and stored at ambient temperatures. Ambient temperature refers to the surrounding environmental temperature of the sample. For example, the preservative may allow the storage of the sample at temperatures between 4 and 37 degrees Celsius (e.g., 10 to 30 degrees Celsius) or up to 55 degrees Celsius, as well as at refrigerated or frozen temperatures. The preservative may be provided in a sample container prior to addition of the saliva, thereby providing ease of use.

The preserved sample may be an unprocessed sample (other than addition of the preservative). For example, the preserved sample may not have undergone one or more, or all, of the following processing steps: storage on ice, freezing, refrigeration, centrifugation, removal of supernatant, discarding of pellet, and aliquoting of supernatant. Preserved samples allow for the fluid to be stored (before undergoing metabolite level detection assays) without the need for cold chain processing. The preserved sample may allow for prolonged storage of the sample without significant degradation of metabolites that would interfere with detection, diagnosis and/or treatment. For example, the sample may be preserved for 24 hours, 48 hours, 72 hours, 5 days, 1 week, 2 weeks, 30 days, 1 month, or 1 year without significant metabolite degradation.

The metabolite biomarkers are also compatible with traditional cold chain processing. Therefore, in another embodiment, the sample for use in the invention is a processed sample. The processed sample may undergo one or more, or all, of the following processing steps: storage on ice, freezing, refrigeration, centrifugation, removal of supernatant, discarding of pellet, and aliquoting of supernatant.

Detection Methods

The metabolites galactose, imidazole, acetone, creatinine, and 5-aminopentanoate, and proprionate have been found by the inventors to be predictive biomarkers for diagnosing AD and MCI. The metabolites acetone, creatinine, and 5-aminopentanoate, and proprionate can also be used to diagnose AD and to distinguish between AD and MCI.

The sample can undergo detection for galactose, imidazole, acetone, creatinine, and 5-aminopentanoate, and proprionate levels according to known techniques for metabolite level detection in a biological sample. The metabolites may be assayed individually, in combination, or by high-throughput methods. Preferred methods are reliable, sensitive and specific for the particular metabolite biomarkers of the invention. The skilled artisan will recognize which detection methods are appropriate based on the sensitivity of the detection method and the abundance of the target metabolite.

In some embodiments, the levels of metabolite are detected by nuclear magnetic resonance spectroscopy (¹H-NMR). In other embodiments, the levels of metabolite are detected by liquid chromatography/gas chromatography coupled with mass spectrometry or using HPLC and UV detection modalities.

Companion Diagnostics

In some embodiments, companion diagnostics may be performed and combined with the biomarker diagnostics of the present invention in advance of, simultaneous with, or to confirm or follow-up the biomarker-based diagnosis. In some embodiments, the companion diagnostic may be evaluation of the symptoms and/or history of the patient suspected of having AD. For example, the patient may have severe symptoms, such as severe memory loss or behavioral abnormalities. A symptom score may be produced based on one or more of symptoms of AD. In some embodiments, the patient is diagnosed with AD (a) if the level of one or more of the metabolites galactose, imidazole, creatinine, and 5-aminopentanoate, and/or proprionate in a sample from the patient is at a different level than a statistically valid threshold and (b) if the companion diagnostic also indicates AD.

One aspect of the invention is a method of providing medical services for a human patient suspected of having or having AD, the method comprising: requesting a sample from and diagnostic information about the patient, wherein the diagnostic information is a level of each of the metabolites galactose, imidazole, creatinine, and 5-aminopentanoate, and/or proprionate in the sample; evaluating the symptoms or history of the patient; and diagnosing the patient with AD when (a) the levels of one or more of the metabolites galactose, imidazole, creatinine, and 5-aminopentanoate, and/or proprionate in the sample are at a different level than a statistically validated threshold for the respective metabolite, and (b) one or more of the patient's symptoms, or history indicate AD. For example, the method may include ordering a diagnostic test for AD based on the levels of metabolites acetone, creatinine, and 5-aminopentanoate, and proprionate.

In another aspect, the metabolite biomarker testing described herein may be performed in conjunction with, before or after “companion” cognitive testing for AD. For example, such cognitive tests may include one or more of: a memory test, a problem solving test, an attention test, a counting test, and a language abilities test. In one example, if both the diagnostic biomarker test and the companion test are indicative or AD, then the patient is treated for AD.

Statistical Methods

In some embodiments, a “statistically validated threshold” is used to diagnose a subject with AD or MCI. In other embodiments, the statistically validated threshold may also be used to diagnose a subject with AD or MCI or to distinguish between AD and MCI. The statistically validated threshold is based on a data set with metabolite level data for control samples and patient samples. The control population may be defined as subjects that do not have AD, do not have MCI, subjects that have no known symptoms of AD or no known symptoms of MCI, or more than one of these groups together. Various control populations are described herein. Either control group, or a combined control group can be used interchangeably with the methods of the invention, including calculating the statistically validated threshold. The statistically validated thresholds are related to the values used to characterize the level of the specific metabolites in the sample obtained from the subject or patient. Thus, if the level of the metabolite is an absolute value, then the control value is also based upon an absolute value.

The statistically validated thresholds can take a variety of forms. For example, a statistically validated threshold can be a single cut-off value, such as a median or mean. Or, a statistically validated threshold can be divided equally (or unequally) into groups, such as low, medium, and high groups, the low group being individuals least likely to have AD and the high group being individuals most likely to have AD.

Statistically validated thresholds, e.g., mean levels, median levels, or “cut-off” levels, may be established by assaying a large sample of individuals in the select population (patients and controls) and using a statistical model such as the predictive value method for selecting a positivity criterion or receiver operator characteristic curve that defines optimum specificity (highest true negative rate) and sensitivity (highest true positive rate). A “cutoff value” may be separately determined for the level of each specific metabolite assayed. Statistically validated thresholds also may be determined according to the methods described in the Examples hereinbelow.

The levels of the assayed metabolites in the patient sample may be compared to single control values or to ranges of control values. In one embodiment, the specific metabolites in a sample from a patient (e.g., a patient having or suspected of having AD) are present at a different level (e.g., at an elevated level) compared to the same specific metabolites in control samples from subjects that do not have AD when the level of the specific metabolites in the patient sample is elevated (greater than) as compared to the statistically validated threshold (e.g., mean concentration) for the control samples. For example, a metabolite is present at a different (e.g., elevated) level when the level of the metabolite in a sample is at least 1.1×, at least 1.2×, at least 1.25×, at least 1.3×, at least 1.4×, at least 1.5×, at least 1.6×, at least 1.7×, at least 1.75×, at least 1.8×, at least 1.9×, at least 2×, at least 2.1×, at least 2.2×, at least 2.25×, at least 2.3×, at least 2.4×, at least 2.5×, at least 3×, at least 3.5×, at least 4×, at least 4.5×, at least 5×, at least 6×, at least 7×, at least 8×, at least 9×, or at least 10×, greater than the statistically validated threshold (e.g., mean concentration) for the respective metabolite in the control samples.

In some embodiments, the methods comprise diagnosing the patient with AD when the levels of two or more of the metabolites imidazole, acetone, creatinine, 5-aminopentanoate, and proprionate in the sample are at an elevated level as compared to a statistically validated threshold for the metabolites imidazole, acetone, creatinine, and 5-aminopentanoate, and proprionate; for example, the levels of two or more of the metabolites, imidazole, acetone, creatinine, and 5-aminopentanoate, and proprionate in the sample is at a level at least 1.1 times (e.g., at least 1.1×, at least 1.2×, at least 1.25×, at least 1.3×, at least 1.4×, at least 1.5×, at least 1.6×, at least 1.7×, at least 1.75×, at least 1.8×, at least 1.9×, at least 2×, at least 2.1×, at least 2.2×, at least 2.25×, at least 2.3×, at least 2.4×, at least 2.5×, at least 3×, at least 3.5×, at least 4×, at least 4.5×, at least 5×, at least 6×, at least 7×, at least 8×, at least 9×, or at least 10×) greater than a statistically validated threshold for the respective metabolite.

If the level of a specific metabolite (or metabolites) in the patient sample is present at a different level than its statistically validated threshold, then the patient is more likely to have AD than are individuals with levels comparable to the statistically validated threshold. In another embodiment, if the level of a specific metabolite or metabolites in the patient sample is present at a different level than its statistically validated threshold, then the patient is more likely to have MCI than are individuals with levels comparable to the statistically validated threshold. The extent of the difference between the subject's levels and statistically validated thresholds is also useful for characterizing the extent of the risk and thereby, determining which individuals would most greatly benefit from certain therapies, e.g., aggressive therapies. In those cases, where the statistically validated threshold ranges are divided into a plurality of groups, such as statistically validated threshold ranges for individuals at high risk of AD, average risk of AD, and low risk of AD, the comparison involves determining into which group the subject's level of the relevant risk predictor falls.

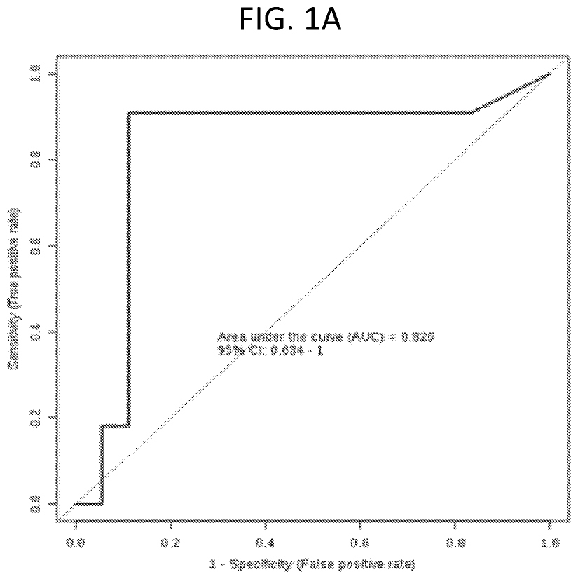

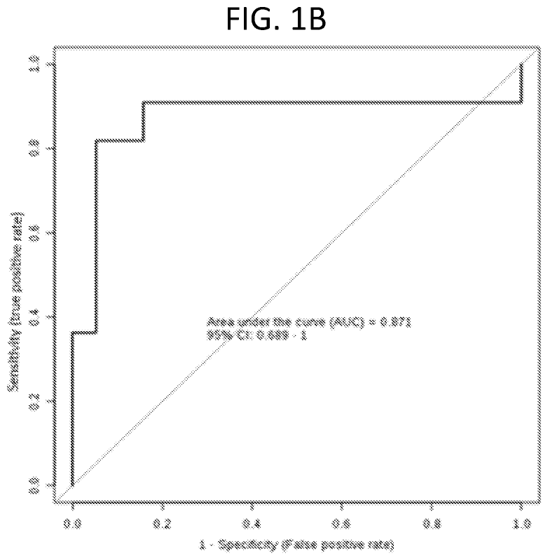

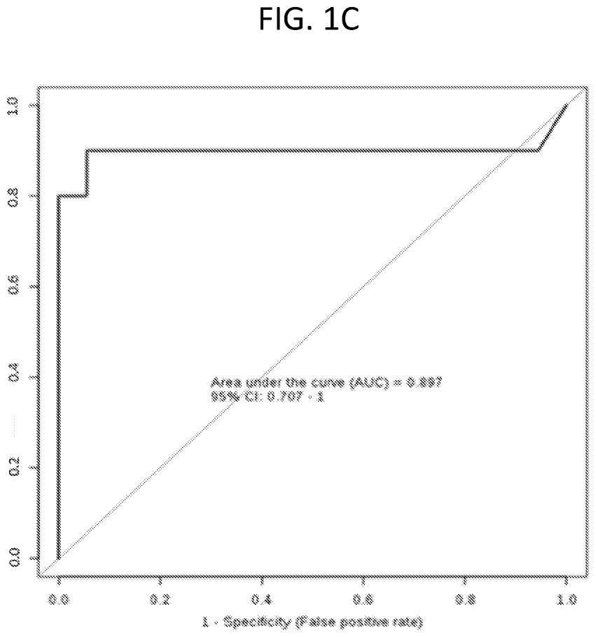

In some embodiments, the metabolite levels are used to diagnose and/or treat AD or MCI. For example, as shown in FIGS. 1A-1C and Table 3, the metabolite expression levels of galactose, imidazole, acetone, proprionate, creatinine, and 5-aminopentanoate for subjects with no MCI, no AD, AD, or MCI may each be differentiated from each other. Therefore, a statistically validated threshold may be used based on the levels of these metabolites in a data set to diagnose MCI or AD or to distinguish between AD and MCI.

In some embodiments, the metabolite levels are used to diagnose and/or treat AD or to distinguish AD from MCI. For example, as shown in FIGS. 1A-1C and Table 3, the metabolite expression levels of acetone, creatinine, proprionate and 5-aminopentanoate for subjects with no AD, AD and MCI may each be differentiated from each other. Therefore, a statistically validated threshold may be used based on the levels of these metabolites in a data set to diagnose AD or to distinguish between AD and MCI.

In some embodiments, the metabolite levels are used to diagnose MCI. For example, as shown in FIGS. 1A-1C and Table 3, the metabolite expression levels of galactose, imidazole, and acetone for subjects with no MCI and MC may be differentiated from each other. Therefore, a statistically validated threshold may be used based on the levels of these metabolites in a data set to diagnose MCI.

In some embodiments, the methods further comprise diagnosing the patient with AD when the levels of the metabolites acetone, creatinine, proprionate and 5-aminopentanoate in the sample are at a different level (e.g., an elevated level) than a statistically validated threshold for the metabolites acetone, creatinine, proprionate and 5-aminopentanoate. In some embodiments, the methods further comprise diagnosing the patient with MCI when the levels of the metabolites galactose, imidazole, and acetone in the sample are at a different level (e.g., an elevated level) than a statistically validated threshold for the metabolites galactose, imidazole, and acetone.

Risk Scores

In some embodiments, a risk score is used to diagnose a subject with AD. In some embodiments, a risk score is used to diagnose and/or treat a subject with MCI. For example, one aspect of the invention comprises obtaining a saliva sample from the human patient, wherein the saliva sample includes the metabolites acetone, creatinine, proprionate and/or 5-aminopentanoate; detecting a level of one or more of the metabolites acetone, creatinine, proprionate and 5-aminopentanoate in the saliva sample; determining a risk score based on the levels of the metabolites acetone, creatinine, proprionate and 5-aminopentanoate in the saliva sample; and diagnosing the patient with AD based on the risk score. The risk score may be a value from 0 to 1, 0-10, 0-100, 1-10, 1-100, −1 to 1, etc. The risk score is calculated based on a data set comprising known control samples (no AD and/or no MCI) and patient samples (AD and/or MCI). For example, the data set includes information for each patient or control sample about whether or not the sample is from a patient with a positive AD diagnosis, and may also differentiate whether the sample is from a subject with a MCI diagnosis or no AD.

The risk score model (used to calculate a risk score) may be generated using an algorithm. In some embodiments, the algorithm is a machine-learning algorithm. The machine learning algorithm is able to analyze data from a large data set, e.g., a training data set, and analyze trends in the data to create a model for calculating a risk score. Many machine-learning algorithms may be appropriate for creating a risk score model using a data set. Machine learning algorithms are of two types: classification and regression algorithms. Classification algorithms were generally found to give better specificity for the data set but regression algorithms can also be used successfully. There are many categories of and particular examples of classification algorithms that can be used with the data set. For example, ensemble, Bayesian, decision tree, and neural network algorithms can be used.

To create the risk score model, the machine learning algorithm is trained on a data set. For example, the diagnosis data (yes AD or no AD) and metabolite level data (concentration or expression level) from each patient and control sample can be used to create a plurality of decision trees. The plurality of decision trees can then be used as a model for calculating a risk score. Each decision tree can lead to an outcome of 0 or 1, depending on whether the individual decision tree would find a negative (0) or positive (1) diagnosis based on the metabolite levels in a new sample. The risk score can be calculated as an average of these outcomes from all of the decision trees generated by the machine-learning algorithm.

The decision trees may be generated from random subsets of the data set. The random subset may be defined as the metabolite levels from one AD patient sample, one MCI control sample and one no AD control sample.

Methods of Diagnosis and Treatment

Current AD therapies are initiated only after diagnosis; their modest benefit may be partly explained by the fact that irreversible brain damage already may have occurred by the time the first signs and symptoms of AD are recognized as dementia. The present biomarkers for MCI and AD will facilitate diagnosis and treatment at the earliest stages of the neurodegenerative process. And in combination with obtaining a patient's results from this biomarker panel, a clinician can determine whether and how to treat the patient for AD, and may do so at an earlier stage of the development of the disease. Such treatments for AD include, but are not limited to, drug therapies, such as, acetylcholinesterase inhibitors (rivastigmine, galantamine, or donepezil) and memantine, alone or in combination. Other treatments for a patient with AD or MCI may include cognitive exercises (e.g. singing or playing music, doing arts and crafts, reading, doing puzzles, cooking, and organizing). Further, a patient's results for the present biomarker panel can be used by the clinician in combination with cognitive testing and/or supportive counseling to the patient and family to help them cope.

The inventive biomarkers will not only help clinicians recognize AD or MCI in its earliest symptomatic stages, but are important when used in connection with disease-modifying therapies. However, for such treatments to be efficacious, an early diagnosis of AD or MCU is important. Thus, the inventive biomarkers facilitate the identification of people at greatest risk of developing AD or MCI sufficiently early so that treatment can be instituted before permanent brain damage has occurred.

In some embodiments, if a patient is diagnosed with AD or MCI according to one or more of the above-described diagnostic methods, then the patient is treated for AD or MCI. The patient may be treated with any known treatments for AD or MCI. In some embodiments, the treatment for AD or MCI includes an acetylcholinesterase inhibitor or an N-methyl-D-aspartate receptor (NMDAR) antagonist, or a combination thereof.

One embodiment is a method of treating Alzheimer's Disease (AD) in a human patient, the method comprising: obtaining a sample from the human patient, wherein the sample consists of one or more of the metabolites selected from galactose, imidazole, acetone, creatinine, and 5-aminopentanoate; detecting a level of one or more of the metabolites acetone, creatinine, and 5-aminopentanoate in the sample; and administering a therapeutically effective amount of a treatment for AD to the patient when the level of one or more of the metabolites galactose, imidazole, acetone, creatinine, and 5-aminopentanoate in the sample is at a different level than a statistically validated threshold for the respective metabolite.

Another embodiment is a method of treating a human patient at risk for developing Alzheimer's Disease (AD), the method comprising: obtaining a sample from the human patient, wherein the sample consists of one or more of the metabolites selected from galactose, imidazole, acetone, creatinine, and 5-aminopentanoate; detecting a level of the one or more metabolites galactose, imidazole, acetone, creatinine, and 5-aminopentanoate in the sample; and administering a therapeutically effective amount of a treatment for AD to the patient to slow or prevent the onset of AD symptoms when the level of one or more of the metabolites galactose, imidazole, acetone, creatinine, and 5-aminopentanoate in the sample is at a different level than a statistically validated threshold for the respective metabolite.

A further embodiment is a method of treating Alzheimer's Disease (AD) in a human patient, the method comprising: obtaining a sample from the human patient, wherein the sample consists of one or more of the metabolites selected from galactose, imidazole acetone, creatinine, and 5-aminopentanoate; requesting testing for a level of one or more of the metabolites galactose, imidazole acetone, creatinine, and 5-aminopentanoate in the sample; and administering a therapeutically effective amount of a treatment for AD to the patient when the level of one or more of the metabolites galactose, imidazole acetone, creatinine, and 5-aminopentanoate in the sample is at a different level than a statistically validated threshold for the respective metabolite. A physician, physician's assistant, nurse or other associated health care provider may obtain the sample, request the testing, and/or administer the therapeutically effective amount of the treatment for AD.

Another aspect of the invention is a method of treating Alzheimer's Disease (AD) in a human patient, the method comprising: obtaining a sample from the human patient, wherein the sample consists of two or more of acetone, creatinine, and 5-aminopentanoate, and proprionate; detecting a level of two or more of acetone, creatinine, and 5-aminopentanoate, and proprionate in the sample; and administering a therapeutically effective amount of a treatment for AD to the patient when the level of two or more of acetone, creatinine, and 5-aminopentanoate, and proprionate in the sample are at a different level than a statistically validated threshold for the respective metabolite.

A further invention is a method of treating a mild cognitive impairment (MCI) in a human patient, the method comprising: obtaining a sample from the human patient, wherein the sample consists of one or more of the metabolites selected from galactose, imidazole, and acetone; detecting a level of one or more of the metabolites galactose, imidazole, and acetone in the sample; and administering a therapeutically effective amount of a treatment for MCI to the patient when the level of one or more of the metabolites galactose, imidazole, and acetone in the sample is at a different level than a statistically validated threshold for the respective metabolite.

In some aspects, the AD symptoms may be a memory loss or a non-memory loss, or combinations thereof. Non-memory losses may be confusion; impaired language, learning, reasoning, judgment, or coping; or personality changes. The treatment for AD may result in an improvement, a stabilization, or a slowed decline in one or more AD symptoms. In some aspects, the MCI symptoms may be a memory loss or a non-memory loss, or combinations thereof; and non-memory losses may be confusion; impaired language, learning, reasoning, judgment, or coping; or personality changes. The treatment for MCI may result in an improvement, a stabilization, or a slowed decline in one or more MCI symptoms.

Using the present method, treatments for AD or MCI may include the administration of oral pharmacologic agents. For example, the oral pharmacologic agents may be an acetylcholinesterase inhibitor, such as, rivastigmine, donepezil, or galantamine. The treatment for AD or MCI also may comprise administration of one or more oral pharmacologic agents that is an NMDAR antagonist, such as, memantine. The treatment for AD or MCI also may include a combination of one or more acetylcholinesterase inhibitors and one or more NMDAR antagonists.

Rivastigmine or rivastigmine tartrate (referred to herein as “rivastigmine”) is a CNS-selective carbamate cholinesterase (ChE) inhibitor that is administered orally or transdermally for the treatment of Alzheimer's Disease. Rivastigmine is a potent, selective inhibitor of brain acetylcholinesterase (AChE) and butylcholinesterase (BChE) and is considered a pseudo-irreversible inhibitor of AChE. For example, rivastigmine currently is marketed under the brand name “Exelon” or “Exelon Patch.”

Donepezil or donepezil hydrochloride (referred to herein as “donepezil”) is a piperidine-type reversible cholinesterase inhibitor that is administered orally for the treatment of AD. Donepezil selectively inhibits acetylcholinesterase (AChE), the enzyme responsible for the destruction of acetylcholine, and improves the availability of acetylcholine. For example, donepezil currently is marketed under the brand name “Aricept.”

Galantamine or galantamine hydrobromide (referred to herein as “galantamine”) is a tertiary alkaloid that is administered orally (immediate-release tablets, extended-release capsules, or solution) for the treatment of AD. Galantamine is considered a reversible inhibitor of acetylcholinesterase (AChE). For example, galantamine currently is marketed under the brand names “Razadyne” and “Reminyl.”

Memantine or memantine hydrochloride (referred to herein as “memantine”) is an antagonist at N-methyl-D-aspartate (NMDA) receptors that partially blocks excitatory glutamate activation. Memantine is administered orally (immediate-release tablets, extended-release capsules, solution) for the treatment of AD. For example, memantine currently is marketed under the brand names “Namenda” and “Namenda XR.”

In some embodiments, the treatment for AD or MCI is the oral administration of a combination of an acetylcholinesterase inhibitor and an NMDA receptor antagonist. For example, a capsule currently marketed under the brand name “Namzaric” includes a combination of donepezil hydrochloride and memantine hydrochloride.

In some embodiments, if a patient is diagnosed with MCI according to the above-described diagnostic methods, then the patient is treated for MCI. The treatments may include any of the treatments described above, or a combination thereof.

Kits

Another embodiment of the present invention is a kit for diagnosing AD. Kits that allow for the targeted measure of the metabolites galactose, imidazole, acetone, creatinine, 5-aminopentanoate, and/or proprionate would reduce both overall cost and turn-around time for a diagnosis of AD or MCI.

In one embodiment, a biomarker panel is used to diagnose AD by detecting acetone, creatinine, 5-aminopentanoate, and/or proprionate levels in the sample. The inventive kit for diagnosing AD may include (a) an NMR assay for detecting the metabolite levels; (b) a container for the sample; and (c) instructions for the method of detection. The kit may further comprise a preservative for the sample. In some embodiments, the preservative is contained within the container in the kit.

In one embodiment, the present diagnostic methods and kits are useful for determining if and when medical treatments and therapeutic agents that are targeted at treating AD should or should not be prescribed for an individual patient. Such medical treatments and therapeutic agents are discussed above, and will be ordered by or prescribed by a physician (or other healthcare provider) based on results of the detection of the metabolites acetone, creatinine, and 5-aminopentanoate, and proprionate.

### EXAMPLES

### Example 1: Materials and Methods

Patient Characteristics: Study subjects were recruited from an academic geriatric practice that is heavily focused on memory disorder.

TABLE 2
Characteristics of the study population.

| | Normal | MCI | AD |
|---|---|---|---|
| Age (mean ± SD) | 82 ± 8 | 83 ± 5 | 85 ± 7 |
| BMI (mean ± SD) | 28.8 ± 8.9 | 25.1 ± 3.0 | 24.3 ± 7.1 |
| CDR (mean ± SD) | 0 | 0.5 ± 0.7 | 1.2 ± 0.8 |
| Years since diagnosis | N/A | 2 ± 2 | 3 ± 2 |
| (mean ± SD) | | | |
| MMSE (mean ± SD) | 29 ± 1 | 28 ± 2 | 20 ± 6 |
| M/F | 4/8 | 3/5 | 3/6 |

All subjects underwent the following cognitive assessments: a) clinical dementia rating scale (CDR), b) mini-mental status examination (MMSE), c) logical memory test, d) digit span forward and backward, e) category fluency test, f) ordering test, g) trails A&B and finally geriatric depression scale.

Samples collection: Human saliva samples were collected from adult volunteers (12 Controls, 8 MCI sufferers and 9 AD patients). The subjects were instructed to refrain from eating, drinking, smoking or using oral hygiene products for at least 1 hour prior to saliva collection. The subjects rinsed their mouth with water for 5 minutes and were instructed to spit into 50-cc Falcon tubes. The subjects were reminded not to cough up mucus. The saliva samples were centrifuged at 2600×g for 35 minutes at 4° C. to remove any sedimentary material. Subsequently, aliquots of the supernatant were stored in Eppendorf tubes at −80° C. until analyzed.

Sample Preparation: Samples thawed at room temperature were filtered through 3-kDa cut-off centrifuge filter units (Amicon Micoron YM-3; Sigma-Aldrich, St. Louis, Mo.) to remove any proteins. Sample preparation was completed following the protocol presented by Dame et al. [Metabolomics 11, 1864-1883. (2015)]. Samples were analyzed in a randomized order and maintained at 4° C. prior to analysis using the state-of-the-art SampleJet (Bruker) automated sample changer.

NMR Analysis: All ¹H-NMR experiments were recorded at 300.0 (+0.05) K on a Bruker Avance III HD 600 MHz spectrometer (Bruker-Biospin, USA) operating at 600.13 MHz equipped with a 5 mm TCI cryoprobe. Data collection was conducted as previously described by Ravanbakhsh et al [PLoS One 10, e0124219 (2015)]. The singlet produced by the DSS methyl groups was used as an internal standard for chemical shift referencing (set to 0 ppm, concentration 500 uM) and for quantification. All ¹H-NMR spectra were processed and analyzed using the Chenomx NMR Suite Professional Software package version 8.1 (Chenomx Inc, Edmonton, AB).

Statistical Analysis: Normalized and Pareto scaled data were analyzed using logistic regression to generate optimal predictive models for MCI and AD. Prior to logistic regression analysis unsupervised PCA was performed to make sure that no outlier was incorporated into the analysis. Significant metabolites subsets were generated using Random Forest analysis. Subsequently, stepwise variable selection was utilized to optimize prediction model components via 10-fold cross-validation. The area under the receiver operating characteristics curve (AUROC or AUC) was calculated using previously described techniques. [Xia J, et al., Metabolomics 9, 280-299 (2013)]. Sensitivity and specificity values were also calculated for each model.

### Example 2: Results

Logistic regression models were used for the detection of MCI and AD. The following metabolites: (i) galactose, imidazole and acetone; (ii) creatinine and 5-aminopentanoate and (iii) propionate and acetone were used for distinguishing control vs. MCI; MCI vs. AD; and control vs. AD, respectively. Using logistic regression modelling, statistically significant prediction of MCI and AD from controls and MCI from AD were achieved.

The logistic regression models are: logit(π)=β0+β0X1+β2X2+. . . +βkXk, where π is the probability of the proportion of case in a group, and Xi is the metabolite concentrations as k covariates.

The descriptive characteristic for the ROC and the logistic regression analysis based on metabolites of interest are shown FIGS. 1A-1C, and Table 3. Table 3 shows the results for the pairwise logistic regression modeling for healthy control vs MCI sufferers, MCI sufferers vs. AD patients, and healthy controls vs. AD patients.

TABLE 3

| | | | | | | | | Concentration |
|---|---|---|---|---|---|---|---|---|
| Models | Coefficients | Estimate | Std. Error | Z | p-value | Odds Ratio | | Change |
| MCI vs | (Intercept) | 0.384 | 2.437 | 0.158 | 0.875 | — | | — |
| Control | Galactose | 5.897 | 3.801 | 1.551 | 0.121 | 0.09 | (0.00-0.72) | Down |
| | Imidazole | −14.714 | 11.792 | −1.248 | 0.212 | 5.05 | (0.66-164.40) | Up |
| | Acetone | 2.049 | 6.789 | 0.302 | 0.763 | 2.40 | (0.86-13.74) | Up |
| AD vs | (Intercept) | 5.435 | 3.59 | 1.514 | 0.13 | — | | — |
| MCI | Creatinine | 14.212 | 8.644 | 1.644 | 0.095 | 2.55 | (1.03-9.93) | Up |
| | 5-Aminopentanoate | 1.204 | 0.78 | 1.545 | 0.122 | 3.62 | (1.33-29.80) | Up |
| AD vs | (Intercept) | 0.083 | 0.654 | 0.127 | 0.899 | — | | — |
| Control | Propionate | −0.74 | 0.349 | −2.12 | 0.034 | 0.48 | (1.95-445.53) | Up |
| | acetone | 0.208 | 0.388 | 0.537 | 0.591 | 2.88 | (1.04-18.46) | Up |

1. A method of treating Alzheimer's Disease (AD) in a human patient, the method comprising:
obtaining a sample from the human patient, wherein the sample consists of one or more of the metabolites selected from galactose, imidazole, acetone, creatinine, and 5-aminopentanoate;
detecting a level of one or more of the metabolites acetone, creatinine, and 5-aminopentanoate in the sample; and
administering a therapeutically effective amount of a treatment for AD to the patient when the level of one or more of the metabolites galactose, imidazole, acetone, creatinine, and 5-aminopentanoate in the sample is at a different level than a statistically validated threshold for the respective metabolite.

2. The method of claim 1, wherein, the treatment is an acetylcholinesterase inhibitor or an N-methyl-D-aspartate receptor (NMDAR) antagonist, or a combination thereof.

3. The method of claim 2, wherein the treatment is an acetylcholinesterase inhibitor selected from the group consisting of rivastigmine, donepezil, and galantamine.

4. The method of claim 2, wherein the treatment is memantine.

5. The method of claim 1, wherein the sample is saliva.

6. The method of claim 1, wherein the one or more of the metabolites are selected from acetone, creatinine, and 5-aminopentanoate, and wherein the different level is an elevated level of the one or more metabolites in the sample than the statistically validated threshold for the respective metabolite.

7. The method of claim 6, wherein the one or more metabolites are creatinine and 5-aminopentanoate.

8. The method of claim 1, further comprising:
performing one or more cognitive tests on the patient; and
treating the patient for AD when (a) the level of one or more of the metabolites galactose, imidazole, acetone, creatinine, and 5-aminopentanoate in the sample is at a different level than a statistically validated threshold for the respective metabolites; and (b) the one or more cognitive tests indicates AD.

9. The method of claim 8, wherein the one of more cognitive tests are selected from the group consisting of a memory test, a problem solving test, an attention test, a counting test, and a language abilities test

10. A method of treating a human patient at risk for developing Alzheimer's Disease (AD), the method comprising:
obtaining a sample from the human patient, wherein the sample consists of one or more of the metabolites selected from galactose, imidazole, acetone, creatinine, and 5-aminopentanoate;
detecting a level of the one or more metabolites galactose, imidazole, acetone, creatinine, and 5-aminopentanoate in the sample; and
administering a therapeutically effective amount of a treatment for AD to the patient to slow or prevent the onset of AD symptoms when the level of one or more of the metabolites galactose, imidazole, acetone, creatinine, and 5-aminopentanoate in the sample is at a different level than a statistically validated threshold for the respective metabolite.

11. The method of claim 10, wherein, the treatment is an acetylcholinesterase inhibitor or an N-methyl-D-aspartate receptor (NMDAR) antagonist, or a combination thereof.

12. The method of claim 11, wherein the treatment is an acetylcholinesterase inhibitor selected from the group consisting of rivastigmine, donepezil, and galantamine.

13. The method of claim 11, wherein the treatment is memantine.

14. The method of claim 10, wherein the sample is saliva.

15. The method of claim 10, wherein the one or more of the metabolites are selected from acetone, creatinine, and 5-aminopentanoate, and wherein the different level is an elevated level of the one or more metabolites in the sample than the statistically validated threshold for the respective metabolite.

16. The method of claim 15, wherein the sample wherein the one or more of the metabolites are creatinine and 5-aminopentanoate.

17. A method of treating Alzheimer's Disease (AD) in a human patient, the method comprising:
obtaining a sample from the human patient, wherein the sample consists of one or more of the metabolites selected from galactose, imidazole acetone, creatinine, and 5-aminopentanoate;
requesting testing for a level of one or more of the metabolites galactose, imidazole acetone, creatinine, and 5-aminopentanoate in the sample; and
administering a therapeutically effective amount of a treatment for AD to the patient when the level of one or more of the metabolites galactose, imidazole acetone, creatinine, and 5-aminopentanoate in the sample is at a different level than a statistically validated threshold for the respective metabolite.

18. The method of claim 17, wherein, the treatment is an acetylcholinesterase inhibitor or an N-methyl-D-aspartate receptor (NMDAR) antagonist, or a combination thereof.

19. The method of claim 18, wherein the treatment is an acetylcholinesterase inhibitor selected from the group consisting of rivastigmine, donepezil, and galantamine.

20. The method of claim 18, wherein the treatment is memantine.

21. The method of claim 17, wherein the sample is saliva.

22. The method of claim 17, wherein the one or more of the metabolites are selected from acetone, creatinine, and 5-aminopentanoate, and wherein the different level is an elevated level of the one or more metabolites in the sample than the statistically validated threshold for the respective metabolite.

23. The method of claim 17, wherein the one or more metabolites are creatinine and 5-aminopentanoate.

24. The method of claim 17, further comprising:
requesting one or more cognitive tests on the patient; and
treating the patient for AD when (a) the level of one or more of the metabolites galactose, imidazole, acetone, creatinine, and 5-aminopentanoate in the sample is at a different level than a statistically validated threshold for the respective metabolites; and (b) the one or more cognitive tests indicates AD.

25. The method of claim 24, wherein the one of more cognitive tests are selected from the group consisting of a memory test, a problem solving test, an attention test, a counting test, and a language abilities test.

26. A method of treating Alzheimer's Disease (AD) in a human patient, the method comprising:
obtaining a sample from the human patient, wherein the sample consists of two or more of acetone, creatinine, and 5-aminopentanoate, and proprionate;
detecting a level of the two or more of acetone, creatinine, and 5-aminopentanoate, and proprionate in the sample; and
administering a therapeutically effective amount of a treatment for AD to the patient when the level of two or more of galactose, imidazole, acetone, creatinine, and 5-aminopentanoate, and proprionate in the sample are at a different level than a statistically validated threshold for the respective metabolites.

27. The method of claim 26, wherein, the treatment is an acetylcholinesterase inhibitor or an N-methyl-D-aspartate receptor (NMDAR) antagonist, or a combination thereof.

28. The method of claim 26, wherein the sample is saliva.

29. A method of treating a mild cognitive impairment (MCI) in a human patient, the method comprising:
obtaining a sample from the human patient, wherein the sample consists of one or more of the metabolites selected from galactose, imidazole, and acetone;
detecting a level of one or more of the metabolites galactose, imidazole, and acetone in the sample; and
administering a therapeutically effective amount of a treatment for MCI to the patient when the level of one or more of the metabolites galactose, imidazole, and acetone in the sample is at a different level than a statistically validated threshold for the respective metabolite.

30. (canceled)

31. The method of claim 29, wherein the sample is saliva.

32. (canceled)
